(19) United States
(12) Patent Application Publication
(10) Pub. No.: US 2007/0123890 A1
(43) Pub. Date: May 31, 2007

(54) TISSUE RETRIEVAL DEVICES AND METHODS

(75) Inventors: Bryce Way, San Jose, CA (US); Minh Tran, Westminster, CA (US); Donald Schomer, Englewood, CO (US); Murray David Solsberg, Englewood, CO (US)

Correspondence Address: CONLEY ROSE, P.C., P. O. BOX 3267, HOUSTON, TX, 77253-3267, US

(73) Assignee: X-STEN, CORP., San Jose, CA (US)

(21) Appl. No.: 11/555,899

(22) Filed: Nov. 2, 2006

Related U.S. Application Data
(60) Provisional application No. 60/733,849, filed on Nov. 4, 2005.

(51) Int. Cl. A61B 17/00

(52) U.S. Cl. 606/79

(57) ABSTRACT

A device for retrieving an excised tissue segment. In an embodiment, the device comprises an elongate body having a central axis and an outer surface. In addition, the device comprises at least two prongs extending from the body in a direction substantially parallel to the central axis. Each prong includes a fixed-end coupled to the body, a free-end distal the body, an inner surface, and an outer surface substantially contiguous with the outer surface of the body. Further, the device comprises a space extending between the inner surfaces of the prongs that accommodates excised tissue.

### CROSS-REFERENCE TO RELATED APPLICATIONS

This application claims benefit of U.S. provisional application Ser. No. 60/733,849 filed Nov. 4, 2005, and entitled “Harpoon Tissue Retrieval Device”, which is hereby incorporated herein by reference in its entirety.

### STATEMENT REGARDING FEDERALLY SPONSORED RESEARCH OR DEVELOPMENT

Not Applicable.

### BACKGROUND

1. Field of the Invention

The present invention relates generally to minimally invasive methods, devices and systems for treating spinal disorders using imaging guidance. More particularly, the present invention relates to devices and methods to reduce stenosis and increase the cross-sectional area of the spinal canal available for the spinal cord. Still more particularly, the present invention relates to devices and methods to percutaneously excise portions of an enlarged ligamentum flavum.

2. Background Information

The vertebral column (spine, spinal column, backbone) forms the main part of the axial skeleton, provides a strong yet flexible support for the head and body, and protects the spinal cord disposed in the vertebral canal, which is formed within the vertebral column. The vertebral column comprises a stack of vertebrae with an intervertebral disc between adjacent vertebra. The vertebrae are stabilized by muscles and ligaments that hold the vertebrae in place and limit the movements of the individual vertebra.

As illustrated in FIG. 1, each vertebra 10 includes a vertebral body 12 that supports a vertebral arch 14. A median plane 210 generally divides each vertebra 10 into two substantially equal lateral sides. Vertical body 12 has the general shape of a short cylinder and is anterior to the vertebral arch 14. The vertebral arch 14 together with vertebral body 12 encloses a space termed the vertebral foramen 15. The succession of vertebral foramen 15 in adjacent vertebra 10 along the vertebral column define the vertebral canal (spinal canal), which contains the spinal cord 28.

Vertebral arch 14 is formed by two pedicles 24 which project posteriorly to meet two laminae 16. The two laminae 16 meet posteriomedially to form the spinous process 18. At the junction of pedicles 24 and laminae 16, six processes arise. Two transverse processes 20 project posterolaterally, two superior articular processes 22 project generally superiorly and are positioned superior to two inferior articular processes 25 that generally project inferiorly.

The vertebral foramen 15 is generally an oval shaped space that contains and protects the spinal cord 28. Spinal cord 28 comprises a plurality of nerves 34 surrounded by cerebrospinal fluid (CSF) and an outermost sheath/membrane called the dural sac 32. The CSF filled dural sac 32 containing nerves 34 is relatively compressible. Posterior to the spinal cord 28 within vertebral foramen 15 is the ligamentum flavum 26. Laminae 16 of adjacent vertebral arches 14 in the vertebral column are joined by the relatively broad, elastic ligamentum flavum 26.

In degenerative conditions of the spine, narrowing of the spinal canal (stenosis) can occur. Lumbar spinal stenosis is often defined as a dural sac cross-sectional area less than 100 mm² or an anterior-posterior (AP) dimension of the canal of less than 10-12 mm for an average male.

The source of many cases of lumbar spinal stenosis is thickening of the ligamentum flavum (e.g., ligamentum flavum 26). Spinal stenosis may also be caused by subluxation, facet joint hypertrophy, osteophyte formation, underdevelopment of spinal canal, spondylosis deformans, degenerative intervertebral discs, degenerative spondylolisthesis, degenerative arthritis, ossification of the vertebral accessory ligaments and the like. A less common cause of spinal stenosis, which usually affects patients with morbid obesity or patients on oral corticosteroids, is excess fat in the epidural space. The excessive epidural fat compresses the dural sac, nerve roots and blood vessels contained therein, and results in back, leg pain and weakness and numbness of the legs. Spinal stenosis may also affect the cervical and, less commonly, the thoracic spine.

Patients suffering from spinal stenosis are typically first treated with exercise therapy, analgesics, and anti-inflammatory medications. These conservative treatment options frequently fail. If symptoms are severe, surgery is required to decompress the spinal cord and nerve roots.

In some conventional surgical procedures to correct stenosis in the lumbar region, an incision is made in the back, and the muscles and supporting structures are stripped away from the spine, exposing the posterior aspect of the vertebral column. The thickened ligamentum flavum is then exposed by removal of a portion of the vertebral arch (e.g., vertebral arch 14), often at the laminae (e.g., laminae 16), covering the back of the spinal canal (laminectomy). The thickened ligamentum flavum ligament can then be excised by sharp dissection with a scalpel or punching instruments, such as a Kerison punch that is used to remove small chips of tissue. The procedure is performed under general anesthesia. Patients are usually admitted to the hospital for approximately five to seven days depending on the age and overall condition of the patient. Patients usually require between six weeks and three months to recover from the procedure. Further, many patients need extended therapy at a rehabilitation facility to regain enough mobility to live independently.

Much of the pain and disability after an open laminectomy results from the tearing and cutting of the back muscles, blood vessels, supporting ligaments, and nerves that occurs during the exposure of the spinal column. Also, because the spine-stabilizing back muscles and ligaments are stripped and detached from the spine during the laminectomy, these patients frequently develop spinal instability post-operatively.

Less invasive techniques offer the potential for reduced post-operative pain and faster recovery compared to traditional open surgery. Percutaneous interventional spinal procedures can be performed with local anesthesia, thereby sparing the patient the risks and recovery time required with general anesthesia. In addition, there is less damage to the paraspinal muscles and ligaments with minimally invasive techniques, thereby reducing pain and preserving these important stabilizing structures.

Various techniques for minimally invasive treatment of the spine are known. Microdiscectomy is performed by making a small incision in the skin and deep tissues to create a portal to the spine. A microscope is then used to aid in the dissection of the adjacent structures prior to discectomy. The recovery for this procedure is much shorter than traditional open discectomies. Percutaneous discectomy devices with fluoroscopic guidance have been used successfully to treat disorders of the disc but not to treat spinal stenosis or the ligamentum flavum directly. Arthroscopy or direct visualization of the spinal structures using a catheter or optical system have also been proposed to treat disorders of the spine including spinal stenosis, however these devices still use miniaturized standard surgical instruments and direct visualization of the spine similar to open surgical procedures. These devices and techniques are limited by the small size of the canal and these operations are difficult to perform and master. In addition, these procedures are painful and often require general anesthesia. Further, the arthroscopy procedures are time consuming and the fiber optic systems are expensive to purchase and maintain.

Hence, it remains desirable to provide simple methods, techniques, and devices for treating spinal stenosis and other spinal disorders without requiring open surgery. It is further desired to provide a system whereby the risk of damage to the dural sac containing the spinal nerves may be reduced.

### SUMMARY OF THE PREFERRED EMBODIMENTS

In accordance with at least one embodiment of the invention, a device for retrieving an excised tissue segment comprises an elongate body having a central axis and an outer surface. In addition, the device comprises at least two prongs extending from the body in a direction substantially parallel to the central axis. Each prong includes a fixed-end coupled to the body, a free-end distal the body, an inner surface, and an outer surface substantially contiguous with the outer surface of the body. Further, the device comprises a space extending between the inner surfaces of the prongs that accommodates excised tissue.

In accordance with another embodiment of the invention, a method for treating stenosis in a spine of a patient comprises inserting a tissue excision device percutaneously into the patient. The tissue excision device comprises a distal cutting end and a through bore. In addition, the method comprises positioning the distal cutting end of the tissue excision device adjacent the region of interest. Further, the method comprises excising a tissue segment from the region of interest with the tissue excision device. Still further, the method comprises inserting a tissue retrieval device percutaneously into the bore of the tissue excision device. The tissue retrieval device comprises an elongate body having a central axis and an outer surface and at least two prongs extending from the body in a direction substantially parallel to the central axis. Moreover, the method comprises advancing the prongs towards the excised tissue segment within the bore of the tissue excision device. In addition, the method comprises grasping the excised tissue segment between the prongs of the tissue retrieval device. Further, the method comprises removing the excised tissue segment from the bore of the tissue excision device.

In accordance with another embodiment of the invention, a kit for performing a procedure on a spine comprises a volume of a contrast medium adapted to be inserted into the epidural space by the insertion member and expanded so as to compress a portion of the thecal sac and provide a safety zone within the epidural space. In addition, the kit comprises a tissue excision device. Further, the kit comprises a tissue retrieval device.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings.

### BRIEF DESCRIPTION OF THE DRAWINGS

For a more complete understanding of the disclosure, reference is made to the accompanying drawings, wherein:

FIG. 1 is cross-section of the spine viewed from the space between two vertebrae, showing the upper surface of one vertebra and the spinal canal with the dural sac and a normal (un-stenosed) ligamentum flavum therein;

FIG. 2 is cross-section of the spine viewed from the space between two vertebrae, showing the upper surface of one vertebra and the spinal canal with the dural sac and a thickened (stenosed) ligamentum flavum therein;

FIG. 3 is an enlarged cross-section of a vertebral foramen, showing a safety zone created by compression of the dural sac;

FIG. 4 is the cross-section of FIG. 3, showing a tissue excision device positioned in the ligamentum flavum according to an ILAMP procedure;

FIG. 5 is the cross-section of FIG. 3, showing a tissue excision tool positioned in the ligamentum flavum according to an alternative MILD procedure;

FIG. 6 is a partial cross-section of the lumbar portion of the vertebral column taken along lines 6-6 of FIG. 1;

FIG. 7 is the cross-section of FIG. 6, showing the orientation of an imaging tool relative to the vertebral column;

FIG. 8 is the cross-section of FIG. 6, showing the orientation of a tissue excision device relative to the vertebral column;

FIG. 9 is a perspective view of the distal portion of an embodiment of a tissue retrieval device;

FIG. 10 is a cross-sectional view of the tissue retrieval device of FIG. 9;

FIG. 11 is an enlarged cross-sectional view of the free-end of the tissue retrieval device of FIG. 9;

FIGS. 12 and 13 are sequential schematic cross-sectional views showing the tissue retrieval device of FIG. 9 retrieving an excised tissue segment from the tissue excision device of FIG. 4 or FIG. 5;

FIG. 14 is a perspective view of another embodiment of a tissue retrieval device; and

FIGS. 15 is a perspective view of the tissue retrieval device of FIG. 14 including a tissue ejector.

### DETAILED DESCRIPTION OF THE PREFERRED EMBODIMENTS

The following discussion is directed to various embodiments of the invention. Although one or more of these embodiments may be presently preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

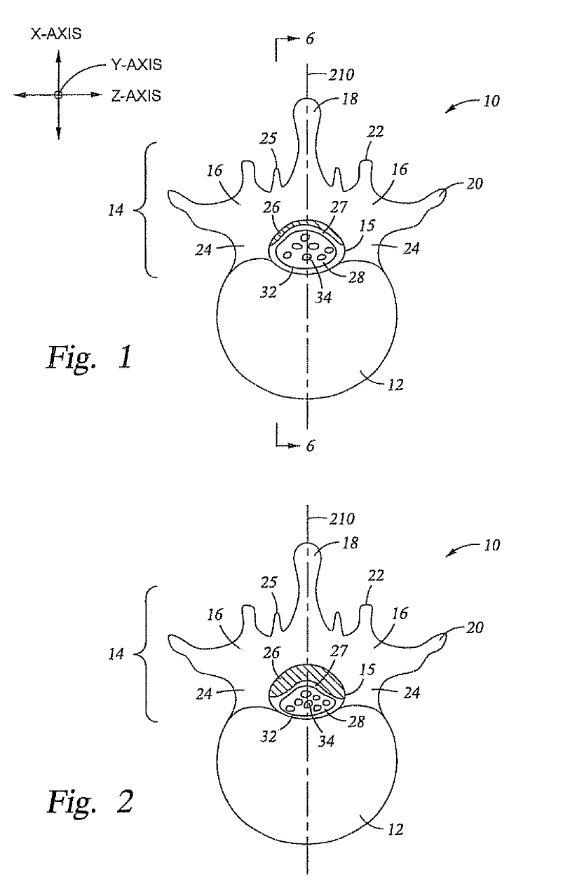

For purposes of this discussion, the x-, y-, and z-axes are shown in FIGS. 1, 3, 6, 7, and 8 to aid in understanding the descriptions that follow. The x-, y-, and z-axes have been assigned as follows. The x-axis is perpendicular to the longitudinal axis of the vertebral column and perpendicular to the coronal/frontal plane (i.e., x-axis defines anterior vs. posterior relationships). The y-axis runs substantially parallel to the vertebral column and perpendicular to the transverse plane (i.e., y-axis defines superior vs. inferior relationships). The z-axis is perpendicular to the longitudinal axis of the vertebral column and perpendicular to the median/midsagittal plane (i.e., z-axis defines the lateral right and left sides of body parts). The set of coordinate axes (x-, y-, and z-axes) are consistently maintained throughout although different views of vertebrae and the spinal column may be presented.

It is to be understood that the median/midsagittal plane passes from the top to the bottom of the body and separates the left and the right sides of the body, and the spine, into substantially equal halves (e.g., two substantially equal lateral sides). Further, it is to be understood that the frontal/coronal plane essentially separates the body into the forward (anterior) half and the back (posterior) half, and is perpendicular to the median plane. Still further, it is to be understood that the transverse plane is perpendicular to both the median plane and coronal plane and is the plane which divides the body into an upper and a lower half.

The Spinal Canal and Spinal Stenosis

Referring again to FIG. 1, vertebral foramen 15 contains a portion of the ligamentum flavum 26, spinal cord 28, and an epidural space 27 between ligamentum flavum 26 and spinal cord 28. Spinal cord 28 comprises a plurality of nerves 34 surrounded by cerebrospinal fluid (CSF) contained within dural sac 32. Nerves 34 normally comprise only a small proportion of the dural sac 32 volume. Thus, CSF filled dural sac 32 is somewhat locally compressible, as localized pressure causes the CSF to flow to adjacent portions of the dural sac. Epidural space 27 is typically filled with blood vessels and fat. The posterior border of the normal epidural space 27 generally defined by the ligamentum flavum 26, which is shown in its normal, non-thickened state in FIG. 1.

FIG. 2 illustrates a case of spinal stenosis resulting from a thickened ligamentum flavum 26. Since vertebral foramen 15 is defined and surrounded by the relatively rigid bone its volume is essentially constant. Thus, thickening of ligamentum flavum 26 within vertebral foramen 15 can eventually result in compression of spinal cord 28. In particular, the thickened ligamentum flavum 26 may exert a compressive force on the posterior surface of dural sleeve 32. In addition, thickening of ligamentum flavum 26 may compress the blood vessels and fat occupying epidural space 27.

Compression of spinal cord 28, particularly in the lumbar region, may result in low back pain as well as pain or abnormal sensations in the legs. Further, compression of the blood vessels in the epidural space 27 that houses the nerves of the cauda equina may result in ischemic pain termed spinal claudication.

In order to relieve the symptoms associated with a thickened or enlarged ligamentum flavum 26, methods, techniques, and devices described herein may be employed to reduce the compressive forces exerted by the thickened ligamentum flavum on spinal cord 28 and the blood vessels in epidural space 27 (e.g., decompress spinal cord 28 and blood vessels in epidural space 27). In particular, compressive forces exerted by the thickened/enlarged ligamentum flavum 26 may be reduced by embodiments of a minimally invasive ligament decompression (MILD) procedure. In some embodiments, the MILD procedure may be performed percutaneously to reduce the size of ligamentum flavum 26 by excising portions of enlarged ligamentum flavum 26. In particular, in some embodiments of the MILD procedure, the ligamentum flavum 26 is accessed, cut and removed ipsilaterally (i.e., on the same side of vertebral arch 14) by a percutaneous caudal-cranial approach. Such an embodiment of the MILD procedure may be described hereinafter as Ipsilateral Approach MILD Procedure (ILAMP).

Creation of a Safety Zone

As shown in FIGS. 1 and 2, ligamentum flavum 26 is posteriorly apposed to spinal cord 28 within vertebral foramen 15. Thus, placement of tools within ligamentum flavum 26 to excise portions of ligamentum flavum 26 creates a risk of for inadvertent damage to the spinal cord 28, dural sac 32, and/or nerves 34. Thus, in preferred embodiments of the procedures described herein, prior to insertion of tissue excision devices into the ligamentum flavum 26, a gap is created between ligamentum flavum 26 and spinal cord 28 to provide a safety zone between ligamentum flavum 26 and spinal cord 28.

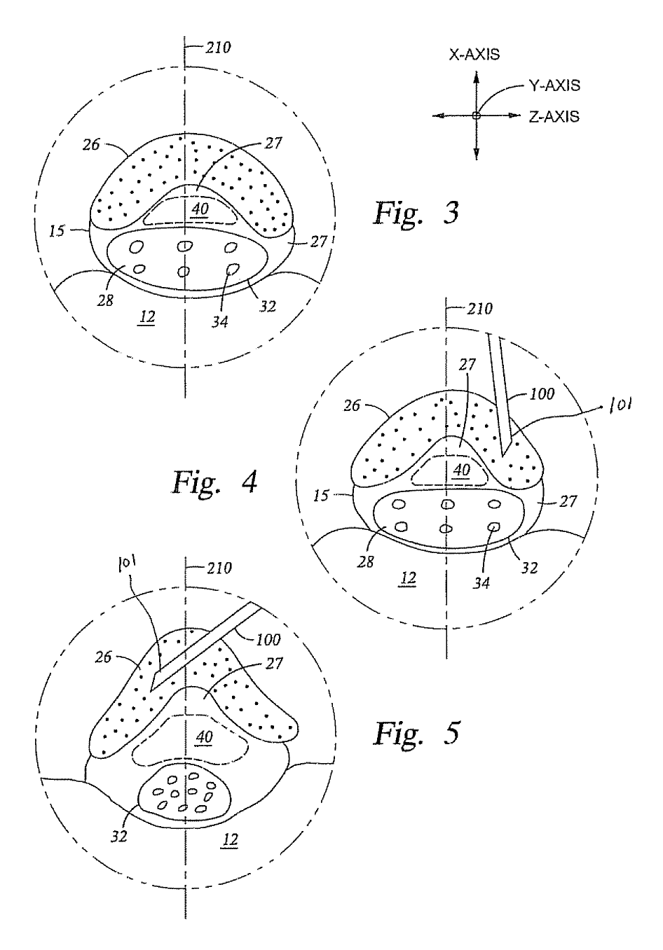

Referring now to FIG. 3, an enlarged cross-sectional view of a vertebral foramen 15 within a vertebra (e.g., vertebra 10) is illustrated. Vertebral foramen 15 includes epidural space 27 and spinal cord 28 containing nerves 34 and CSF within dural sac 32. Further, a thickened/enlarged ligamentum flavum 26 extends into vertebral foramen 15. To reduce the risk of damage to dural sac 32 and spinal cord 28, a safety zone 40 is created between ligamentum flavum 26 and dural sac 32 in the manner described below.

As previously described, spinal cord 28 comprises nerves 34 surrounded by CSF and is contained within dural sac 32. Since more than 90% of the volume of dural sac 32 in the lumbar region is filled by CSF, dural sac 32 is highly compressible. Thus, even when stenosis is causing compression of spinal cord 28, in most cases it is possible to temporarily compress spinal cord 28 further. Thus, according to preferred embodiments, dural sac 32 is further compressed in the region of interest by injecting a fluid or medium into epidural space 27 to create safety zone 40. The fluid may be injected into the epidural space 27 with an insertion member, such as a needle. The presence of the injected fluid comprising safety zone 40 gently applies an additional compressive force to the outer surface of dural sac 32 so that at least a portion of the CSF within dural sac 32 is forced out of dural sac 32 in the region of interest, resulting in safety zone 40 between dural sac 32 and ligamentum flavum 26.

According to some embodiments, dural sac 32 is compressed by injecting a standard radio-opaque non-ionic myelographic contrast medium or other imagable or non-imagable medium into epidural space 27 in the region of interest. This is preferably accomplished with a percutaneous injection. Sufficient injectable fluid is preferably injected to displace the CSF out of the region of interest and compress dural sac 32 to at least a desired degree. The injected medium is preferably substantially contained within the confines of epidural space 27 extending to the margins of the dural sac 32. The epidural space is substantially watertight and the fatty tissues and vascularization in epidural space 27, combined with the viscous properties of the preferred fluids, serve to substantially maintain the injected medium in the desired region of interest. This method for protecting spinal cord 28 column may be referred to hereinafter as “contrast-guided dural protection.”

Referring now to FIGS. 4 and 5, once safety zone 40 has been created, a tissue excision tool or device 100 may be inserted into ligamentum flavum 26. More specifically, the distal cutting end 101 of tissue excision device 100 is inserted into ligamentum flavum 26 in preparation for excising portions of enlarged ligamentum flavum 26. Tissue excision device 100 may comprise any suitable device, tool or instrument for decompressing an enlarged ligamentum flavum 26 and relieving spinal stenosis caused by the enlarged ligamentum flavum. A variety of suitable tissue excision devices, including distal cutting ends, are disclosed in U.S. Application Ser. Nos. 11/193,581, 11/461,036, 60/733,754, 60/733,552, and 11/461,045, each of which is hereby incorporated herein by reference in its entirety.

Although tissue excision device 100 is shown as directly accessing ligamentum flavum 26 in FIGS. 4 and 5 (i.e., without guidance from a cannula or portal), it should be appreciated that tissue excision device 100 may alternatively percutaneously access ligamentum flavum 26 via a cannula or other portal device. For instance, in some embodiments, tissue excision device 100 may be guided by and advanced through a cannula toward the ligamentum flavum 26.

In the embodiment illustrated in FIG. 4, distal cutting end 101 of tissue excision device 100 is inserted and positioned in ligamentum flavum 26 on the same side (ipsilateral) of median plane 210 as tissue excision device 100 percutaneously accesses the patient. Consequently, tissue excision device 100 does not cross median plane 210. However, in the embodiment illustrated in FIG. 5, distal cutting end 101 of tissue excision device 100 is positioned in ligamentum flavum 26 on the opposite side of median plane 210 as tissue excision device 100 percutaneously accesses the patient. Consequently, in this embodiment, tissue excision device 100 crosses median plane 210.

While it is preferred that distal cutting end 101 of tissue excision device 100 remain within ligamentum flavum 26 as shown, the presence of safety zone 40 reduces the likelihood that dural sac 32 will be damaged, even if distal tip 101 of device 100 breaks through the anterior surface of ligamentum flavum 26.

Because the present techniques are preferably performed percutaneously, certain aspects of the methods described herein may be facilitated by imaging. Imaging windows (e.g., a fluoroscopic window of access-FWA) may be employed to aid in performance of all or part of the procedures described herein. For instance, an imaging window may be employed to aid in insertion of tissue excision device 100 into ligamentum flavum 26 as shown in FIGS. 4 and 5. The methods and procedures described herein may be aided by any suitable imaging technology including, without limitation, 2D fluoroscopy, 3D fluoroscopy, CT, MRI, ultrasound or with direct visualization with fiber optic or microsurgical techniques. Stereotactic or computerized image fusion techniques are also suitable. Fluoroscopy is currently particularly well-suited to the techniques disclosed herein since fluoroscopic equipment is relatively safe and easy to use, readily available in most medical facilities, and relatively inexpensive.

In an exemplary procedure using direct biplane fluoroscopic guidance and local anesthesia, epidural space 27 is accessed for injection of contrast media adjacent to the surgical site. If the injected medium is radio-opaque, as are for example myelographic contrast media, the margins of expanded epidural space 27 will be readily visible using fluoroscopy or CT imaging. Thus, safety zone 40 created by the contrast-guided dural compression techniques can reduce the risk of damage to dural sac 32 and spinal cord 28 during MILD procedures to remove or displace portions of ligamentum flavum 26 and/or laminae 16 in order to treat spinal stenosis.

Injectable Medium

If desired, the injected fluid or medium can be provided as a re-absorbable water-soluble gel, so as to better localize safety zone 40 at the site of surgery and reduce leakage of this protective layer from the vertebral/spinal canal. The gel is preferably substantially more viscid and/or viscous than conventional contrast media. In general, a preferred viscid and/or viscous gel tends to remain localized at the desired site of treatment since it does not spread as much as standard liquid contrast media that are conventionally used in epidurography. This may result in more uniform compression of dural sac 32 and less leakage of the contrast medium out of the vertebral/spinal canal. In addition, preferred embodiments of the gel are re-absorbed more slowly than conventional contrast media, allowing for better visualization during the course of the surgical procedure(s).

A standard hydrophilic-lipophilic block copolymer (Pluronic) gel known in the art or other suitable gel may be employed as the injectable medium. The gel preferably has an inert base. In certain embodiments, the gel material is liquid at ambient temperatures and can be injected through a small bore, such as a 27 gauge needle. The gel then preferably becomes viscous when warmed to body temperature after being injected. The viscosity of the gel can be adjusted through the specifics of the preparation. The gel or other fluid is preferably sufficiently viscid and/or viscous at body temperature to compress and protect dural sac 32 in the manner described above and to remain sufficiently present in the region of interest for at least about 30 minutes. Thus, in some embodiments, the injected gel attains a viscosity that is two, three, six or even ten times that of the fluids that are typically used for epidurograms.

In certain embodiments, the injected medium undergoes a reversible change in viscosity when warmed to body temperature so that it can be injected as a low-viscosity fluid, thicken upon injection into the patient, and be returned to its low-viscosity state by cooling. In these embodiments, the injected medium is injected as desired, thickens upon warming, but can be removed by contacting it with a heat removal device, such as an aspirator that has been provided with a cooled tip. As a result of localized cooling, the gel reverts to its initial non-viscous liquid state and can be easily suctioned up the cooled needle or catheter.

In some embodiments, a contrast agent can be included in the gel itself, so that the entire gel mass is imagable. In different embodiments, the contrast agent may be injected first, followed by the desired amount of gel, or vice versa. In the embodiments in which the contrast agent and gel are injected separately, the contrast agent tends to be captured on the surface of the expanding gel mass, so that the periphery of the gel mass is imagable.

An example of a suitable injectable medium, including a contrast agent, having the desired properties is Omnipaque® 240 available from Nycomed, N.Y., which is a commercially available non-ionic iodinated myelographic contrast medium. Other suitable injectable media will be known to those skilled in the art. Because of the proximity to spinal cord 28 and spinal nerves 34, it is preferred not to use ionic media in the injectable medium. The preferred compositions are reabsorbed relatively rapidly after the procedure. Thus any residual gel compression on dural sac 32 after the MILD procedure dissipates relatively quickly. For example, in preferred embodiments, the gel would have sufficient viscosity to compress dural sac 32 for thirty minutes, and sufficient degradability to be substantially reabsorbed within approximately two hours.

The injected medium may further include one or more bioactive agents. For example, medications such as those used in epidural steroid injection (e.g. Depo medrol, Celestone Soluspan) may be added to the epidural gel to speed healing and reduce inflammation, scarring and adhesions. The gel preferably releases the steroid medication slowly and prolongs the anti-inflammatory effect, which can be extremely advantageous. Local anesthetic agents may also be added to the gel. This prolongs the duration of action of local anesthetic agents in the epidural space to prolong pain relief during epidural anesthesia. In this embodiment, the gel may be formulated to slow the reabsorption of the gel.

The above-described injected mediums and gels may also be used for epidural steroid injection and perineural blocks for management of acute and chronic spinal pain. Thrombin or other haemostatic agents can be added if desired, so as to reduce the risk of bleeding.

In some embodiments, the gel may also be used as a substitute for a blood patch if a CSF leak occurs. The gel may also be used as an alternative method to treat lumbar puncture complications such as post-lumbar puncture CSF leak or other causes of intracranial hypotension. Similarly, the gel may be used to patch postoperative CSF leaks or dural tears. If the dural sac were inadvertently torn or cut, then gel could immediately serve to seal the site and prevent leakage of the cerebral spinal fluid.

Ipsilateral Approach for MILD Procedure (ILAMP)

Once safety zone 40 has been created, the margins of epidural space 27 are clearly demarcated by the injected medium and may be visualized radiographically if an imageable medium or contrast agent has been used. As mentioned above, percutaneous procedures can then be performed on ligamentum flavum 26 and/or surrounding tissues, with reduced potential for injuring dural sac 32 and spinal cord 28.

A variety of suitable techniques and devices may be employed to reduce the size of the thickened/enlarged ligamentum flavum 26, thereby decompressing spinal cord 28 as well as blood vessels contained within the epidural space 27. Examples of suitable decompression techniques include without limitation, removal of tissue from ligamentum flavum 26, laminectomy, laminotomy, retraction and anchoring of ligamentum flavum 26, or combinations thereof. In some embodiments, a portion of the enlarged ligamentum flavum 26 is excised using tissue excision device 100 as best shown in FIGS. 4 and 5.

Accessing ligamentum flavum 26 with a tissue excision device 100 may present challenges. For instance, in some conventional approaches to correct stenosis caused by an enlarged ligamentum flavum 26, an incision is made in the back of the patient and then the muscles and supporting structures of the vertebral column (spine) are stripped away, exposing the posterior aspect of the vertebral column. Subsequently, the thickened ligamentum flavum 26 is exposed by removal of a portion of vertebral arch 14, often at lamina 16, which encloses the anterior portion of the spinal canal (laminectomy). The thickened ligamentum flavum 26 can then be excised by sharp dissection with a scalpel or punching instruments. However, this approach is usually performed under general anesthesia and typically requires an extended hospital stay, lengthy recovery time and significant rehabilitation. As another example, some MILD procedures access ligamentum flavum 26 percutaneously by boring a hole through the vertebral arch 14 of vertebra 10, often through a lamina 16. A cannula and/or device 100 may be passed through the bore and/or anchored to the bore to access ligamentum flavum 26 for excision. While such a MILD approach is less invasive and reduces recovery time compared to the procedure just described, such an approach requires the additional step of boring a hole in the posterior of the vertebra 10 of interest. Thus, in some cases it will be preferable to employ a MILD procedure that percutaneously accesses ligamentum flavum 26 without the need to cut or bore through the vertebra.

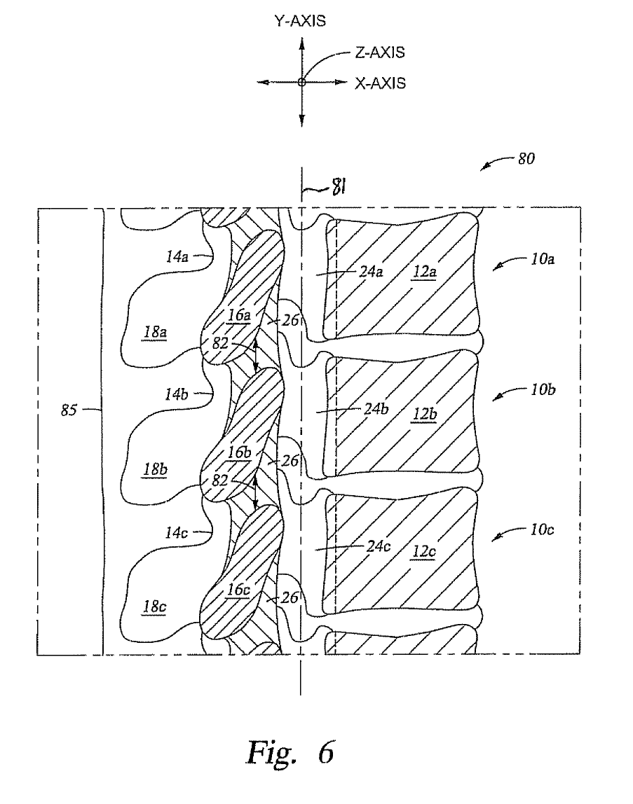

Referring now to FIG. 6, a partial cross-sectional lateral view of a segment of a vertebral column 80 is illustrated. The segment of vertebral column 80 illustrated in FIG. 6 includes three vertebrae 10a, 10b, and 10c. Each vertebrae 10a, 10b, 10c includes a vertebral body 12a, 12b, 12c, that supports a vertebral arch 14a, 14b, 14c, respectively. Vertical body 12a, 12b, 12c is anterior to vertebral arch 14a, 14b, 14c, respectively. Each vertebral arch 14a, 14b, 14c together with vertebral body 12a, 12b, 12c, respectively, encloses a vertebral foramen 15a, 15b, 15c. The succession of vertebral foramen 15a, 15b, 15cin adjacent vertebrae 10a, 10b, 10c define vertebral canal 81 (spinal canal) that runs along the length of vertebral column 80. Vertebral canal 81 contains the spinal cord (not shown in FIG. 5).

As previously described, each vertebral arch 14a, 14b, 14c includes two pedicles that project posteriorly to meet two lamina 16a, 16b, 16c, respectively. In FIG. 6, one pedicle has been removed from each vertebrae 10a, 10b, 10c and thus, only the cross-section of one lamina 16a, 16b, 16c for each vertebrae 10a, 10b, 10c, respectively, is shown. The two lamina meet posteriomedially to form the spinous process 18a, 18b, 18c, respectively.

Lamina 16a, 16b, 16c of adjacent vertebrae 10a, 10b, 10c are connected by ligamentum flavum 26 (shown in cross-section). The relatively elastic ligamentum flavum 26 extends almost vertically from superior lamina to inferior lamina of adjacent vertebrae. In particular, ligamentum flavum 26 originates on the inferior surface of the laminae of the superior vertebra and connects to the superior surface of the laminae of the inferior vertebra. For instance, ligamentum flavum 26 originates on the inferior surface of lamina 16aof superior vertebra 10a and connects to the superior surface of lamina 16b of the inferior vertebra 10b. Thus, ligamentum flavum 26 spans an interlaminar space 82. Interlaminar space 82 is generally the space between laminae of adjacent vertebrae in spinal column 80.

Still referring to FIG. 6, each lamina 16a, 16b, 16c comprises a relatively broad flat plate of bone that extends posteromedially and slightly inferiorly from pedicles 24a, 24b, 24c, respectively. Along the length of vertebral column 80, the lamina 16a, 16b, 16c overlap like roofing shingles, with each lamina substantially parallel to and at least partially overlapping the adjacent inferior lamina. Further, the adjacent substantially parallel laminae are separated by the intervening ligamentum flavum 26 and interlaminar space 82. For instance, lamina 16a is substantially parallel to and partially overlaps adjacent inferior lamina 16b and is separated from lamina 16b by ligamentum flavum 26 and interlaminar space 82.

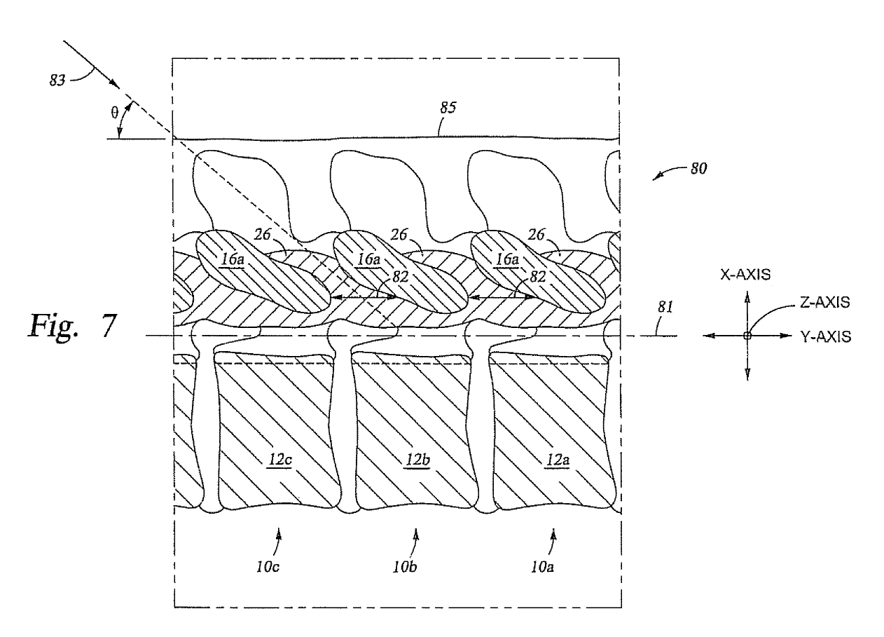

FIG. 7 illustrates vertebral column 80 as it may be oriented with the anterior side positioned down and posterior back surface 85 positioned upward, as may be encountered during a spinal procedure or surgery. In addition, in the embodiment illustrated in FIG. 7, ligamentum flavum 26 is thickened or enlarged, resulting in spinal stenosis. In particular, the anterior portions of enlarged ligamentum flavum 26 extend partially into spinal canal 81, potentially exerting compressive forces on the spinal cord (not shown) that resides within spinal canal 81.

As previously discussed, to relieve compressive forces on the spinal cord and hence relieve the associated symptoms of spinal stenosis, portions of ligamentum flavum 26 may be excised. However, to percutaneously excise portions of ligamentum flavum 26 via minimally invasive techniques, the innate structure of vertebral column 80 and each vertebra may present significant imaging challenges. For instance, lateral imaging windows/views of ligamentum flavum 26 substantially in the direction of the z-axis may be obscured by the various processes of the vertebrae (e.g., transverse processes, superior articular processes, inferior articular processes), the laminae of the vertebra, etc. Further, some anterior-posterior (A-P) imaging windows/views of ligamentum flavum 26 substantially in the direction of the x-axis may also be obscured by the laminae. In particular, in the A-P radiographic imaging planes substantially in the direction of the x-axis, the posterior edges of parallel laminae overlap and obscure ligamentum flavum 26 and interlaminar space 82, particularly the anterior portions of ligamentum flavum 26 and interlaminar space 82 closest to spinal canal 81. However, with an imaging window/view in a plane substantially parallel to the X-Y plane, at an angle θ generally in the direction of arrow 83, and slightly lateral to the spinous process, interlaminar space 82 and ligamentum flavum 26 may be viewed with less obstruction from neighboring laminae. In other words, imaging windows/views generally aligned with arrow 83 (FIG. 7) allow a more direct view of interlaminar space 82 and ligamentum flavum 26 from the posterior back surface with minimal obstruction by the vertebrae, and more specifically the laminae.

Typically, the long axes of the substantially parallel laminae (e.g., laminae 16a, 16,b, 16c) and interlaminar spaces (e.g, interlaminar spaces 82) are generally oriented between 60° and 75° relative to posterior back surface 85. Thus, preferably the imaging means (e.g., x-ray beam, fluoroscopy tube, etc.) is positioned generally in the direction represented by arrow 83, where θ between posterior back surface 85 and the imaging beam is substantially between 60° and 75°. In other words, the imaging means is positioned substantially parallel to the surface of the laminae. The resulting imaging window/view, termed “caudal-cranial posterior view” hereinafter, permits a clearer, more direct, less obstructed view of interlaminar space 82 and ligamentum flavum 26 from the general posterior back surface 85. The caudal-cranial posterior view permits a relatively clear view of interlaminar space 82 and ligamentum flavum 26 in directions generally along the y-axis and z-axis. However, the caudal-cranial posterior view by itself may not provide a clear imaging window/view of interlaminar space 82 and ligamentum flavum 26 in directions generally along the x-axis. In other words, the caudal-cranial posterior view by itself may not provide a clear imaging window or view that can be used to accurately determine the posterior-anterior depth, measured generally along the x-axis, of a device across the ligamentum flavum 26.

Thus, in preferred embodiments, an additional imaging window/view, termed “caudal-cranial posterior-lateral view” hereinafter, is employed to provide a clearer, unobstructed view of interlaminar space 82 and ligamentum flavum 26 in directions generally along the y-axis and z-axis. The caudal-cranial posterior-lateral view is generated by orienting an imaging means generally at an angle θ relative to posterior back surface 85 of the patient and also angling such imaging means laterally in an oblique orientation, revealing a partial lateral view of interlaminar space 82 occupied by ligamentum flavum 26 on the anterior side of the lamina and posterior to the underlying dural sac (not shown) and spinal cord (not shown).

By employing at least one of the caudal-cranial posterior view and the caudal-cranial posterior-lateral views, relatively clear imaging windows/views of the interlaminar space 82 and ligamentum flavum 26 in directions along the x-, y-, and z-axes may be achieved.

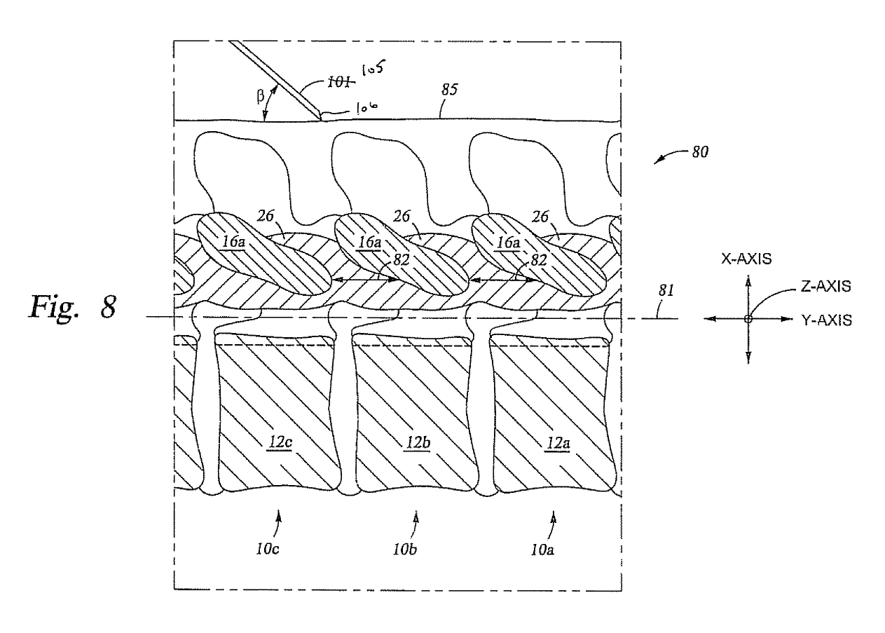

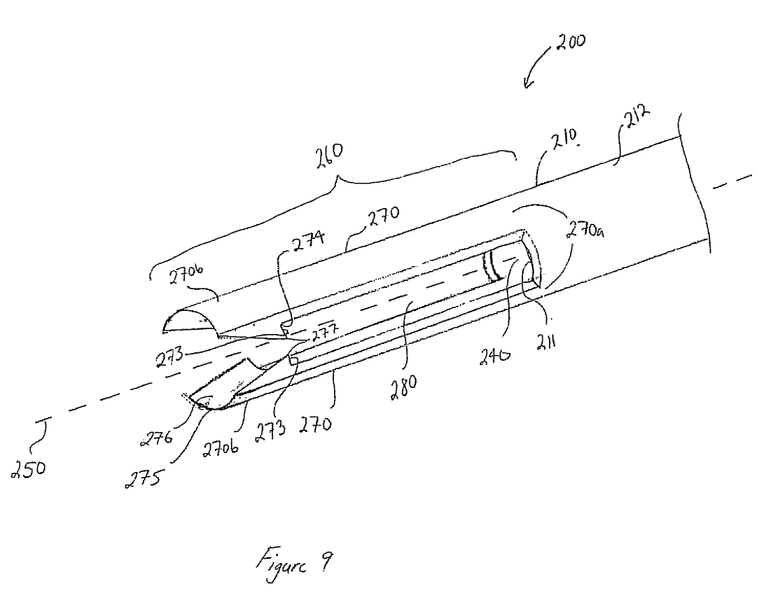

Referring now to FIG. 8, vertebral column 80 and a tissue access instrument 105 including a distal end 106 are illustrated. Tissue access instrument 105 may comprise a tissue excision device (e.g., tissue excision device 100), a cannula, a catheter, or other portal. Once unobstructed imaging windows/views of interlaminar space 82 and ligamentum flavum 26 are established in the manner previously described, tissue access instrument 105 is employed to percutaneously access interlaminar space 82 and ligamentum flavum 26. More specifically, using images of the interlaminar space 82 and ligamentum flavum 26 obtained from the desired direction(s), (e.g., caudal-cranial posterior view and the caudal-cranial posterior-lateral view), tissue access device 105 may be employed to penetrate the skin and soft tissue in the posterior back surface 85 of the patient. In preferred embodiments, the skin entry point for tissue excision device 100 is between 5 and 10 cm inferior (caudal to) the posterior surface of the interlaminar space 82 of interest. For instance, if the portion of ligamentum flavum 26 between lamina 16a and lamina 16b is the area of interest, then tissue excision device 100 may be inserted into the patient's back about 5 to 10 cm inferior to posterior surface 84 of interlaminar space 82.

Referring still to FIG. 8, tissue access device 105 is preferably initially inserted into the posterior tissue and musculature of the patient generally parallel to the longitudinal axis of spinal column 80. In other words, the angle β between the posterior back surface 85 and tissue access device 105 is preferably between 0° and 10° when tissue access device 105 is initially inserted. Further, tissue access device 105 is preferably inserted into the posterior tissue and musculature of the patient on the same side (ipsilateral) of the median plane as the area of interest (e.g., the targeted portion of ligamentum flavum 26), as best seen in FIG. 4. Once tissue access device 105 is inserted into the posterior tissue and musculature of the patient, tissue access device 105 then may be oriented 5° to 90° relative to the posterior back surface 85 in order to create a trajectory across ligamentum flavum 26 in the area of interest. It is to be understood that once tissue access device 105 is inserted into the patient's posterior back surface 85, the ends of tissue access device 105 (e.g., distal end 106) are free to pivot about the insertion location in posterior back surface 85 in the general direction of the y-axis and the z-axis, and may be advanced posteriorly or anteriorly generally in the direction of the x-axis.

Once inserted into the posterior tissue and musculature of the patient, tissue access device 105 can be positioned to provide a trajectory across interlaminar space 82 in the area of interest, generally towards the anterior surface of the lamina superior to the area of interest. For example, if interlaminar space 82 between lamina 16a and lamina 16b is the area of interest, tissue access device 105 is positioned to provide a trajectory that will allow a cutting instrument to be inserted across interlaminar space 82 between lamina 16a and lamina 16b towards the anterior surface of lamina 16a (superior lamina).

By switching between the caudal-cranial posterior view and the caudal-cranial posterior-lateral view, or by viewing both the caudal-cranial posterior view and the caudal-cranial posterior-lateral view at the same time, tissue access device 105 can be advanced to ligamentum flavum 26 in the area of interest with more certainty than has heretofore been present. Once distal end 106 of tissue access device 105 has reached ligamentum flavum 26, portions of ligamentum flavum 26 may be excised with a tissue excision device (e.g., tissue excision device 100) so as to relieve pressure on the spinal nerves. If tissue access device 105 comprises a tissue excision tool, it may be inserted into ligamentum flavum 26 to excise portions of ligamentum flavum 26. However, if tissue access device 105 comprises a cannula or portal, tissue access device 105 will be positioned adjacent or slightly within the ligamentum flavum 26 in the region of interest and a tissue excision device may be advanced through, and guided by, tissue access device 105 toward ligamentum flavum 26. In some embodiments, excision can be performed generally from posterior to anterior across interlaminar space 82 and then laterally along the anterior portion of ligamentum flavum 26 if desired. The actual depth of distal end 106 of tissue access device 105 (or any tissue excision device passing through tissue access device 105) in the general direction of the x-axis may be adjusted with guidance from the caudal-cranial posterior-lateral view and appropriate retraction/advancement of tissue access device 105 and appropriate adjustment of tissue access device 105 between 5° and 90° relative to the posterior back surface 85.

Referring now to FIG. 4, the tip of an exemplary tissue excision device 100 is shown schematically within ligamentum flavum 26. Tissue excision device 100 may be the same device as tissue access device 105, or may be a tool passed through tissue access device 105 if tissue access device 105 is a cannula or portal. In particular, tissue excision device 100 has accessed ligamentum flavum 26 according to the ILAMP method previously described. Thus, device 100 is positioned to excise portions of ligamentum flavum 26 on the same lateral side of median plane 210 as device 100 is percutaneously inserted. In other words, in the view shown in FIG. 4, device 100 is inserted into the body on the right side of median plane 210 and enters ligamentum flavum 26 on the right side of median plane 210 to excise portions of ligamentum flavum 26 on the right side of median plane 210. In FIG. 4, device 100 does not cross median plane 210.

FIG. 5 illustrates an embodiment of an alternative MILD method in which exemplary tissue excision device 100 is positioned to excise portions of ligamentum flavum 26 on the opposite lateral side of median plane 210 as device 100 is percutaneously inserted. More specifically, tissue excision device 100 is inserted into the body on the rights side of median plane 210, enters ligamentum flavum 26 on the right side of median plane 210, but is positioned to excise portions of ligamentum flavum 26 on the left side of median plane 210. In FIG. 5, device 100 crosses median plane 210.

In the manner described, portions of the ligamentum flavum can be excised by a percutaneous MILD procedure. In particular, with the approach described and as best illustrated in FIGS. 4 and 6, ligamentum flavum 26 can be accessed, and portions thereof removed via the interlaminar space on the same lateral side (ipsilateral) of median plane 210 as the entry point for instrument 101 (e.g., a cannula, a tissue excision tool, etc.). This approach may sometimes hereinafter be referred to as an Iplsilateral Approach MILD Procedure (ILAMP).

Tissue Excision Devices

In general, tissue excision device 100 is employed to excise relatively small portions of the stenosed or enlarged ligamentum flavum. By excising several small portions of the ligamentum flavum, the enlarged ligamentum flavum may be decompressed, thereby relieving pressure imposed on the spinal cord and the associated pain and other symptoms. Tissue excision device 100 typically includes a body having a central bore and a distal tip or cutting end adapted to cut a tissue segment (e.g., a segment of an enlarged ligamentum flavum). The cutting end is percutaneously inserted into the patient in the manner previously described and advanced toward the region of interest (e.g., enlarged ligamentum flavum). Once the cutting end of the tissue excision device 100 is positioned adjacent the region of interest, it may be employed to cut segments of tissue from the region of interest. The excised tissue segments are typically retained within the central lumen or bore of tissue excision device 100.

Since the surgical procedures described herein are performed adjacent sensitive tissue (e.g., nerves of the spinal cord), they are preferably performed delicately and with minimal movement of the tools and devices near the sensitive tissues (e.g., tissue excision device 100). Thus, it may be desirable to minimize repositioning of tissue excision device 100, especially the distal tip or cutting end of tissue excision device 100. As used herein, the term “distal” refers to positions that are relatively closer to the region of interest (e.g., the thickened portion of the ligamentum flavum to be decompressed). To further this objective, in some embodiments, tissue excision device 100 may be substantially maintained within or adjacent the area of interest (e.g., enlarged ligamentum flavum) while making repeated excisions of portions of the tissue in the region of interest. In other words, in such embodiments, tissue excision device 100 may not be completely withdrawn from the area of interest and reinserted into the area of interest between each separate excision.

In cases when the distal tip or cutting end of tissue excision device 100 is maintained within the area of interest (i.e., not removed from the patient between each excision), the excised tissue segments may build up within the bore of tissue excision device 100. Excessive build-up of excised tissue within tissue excision device 100 may inhibit or detrimentally impact continued cutting. Thus, between each excision by tissue excision device 100, or at any desired time or interval, excised tissue segments within the bore of tissue excision device 100 are preferably retrieved and removed.

It is to be understood that tissue excision device 100 may comprise any suitable device capable of being employed to safely excise small portions of an enlarged ligamentum flavum. A variety of suitable tissue excision devices are disclosed in U.S. Application Ser. Nos. 11/193,581, 11/461,036, 60/733,754, 60/733,552, and 11/461,045, each of which is hereby incorporated herein by reference in its entirety.

Tissue Retrieval Devices

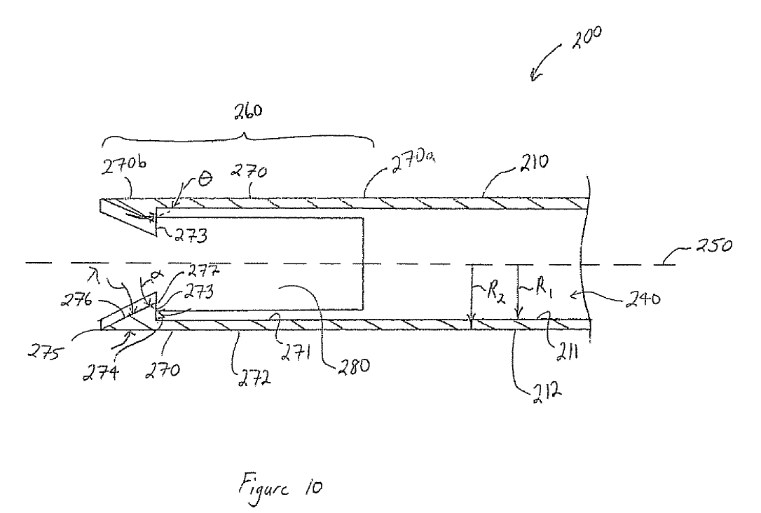

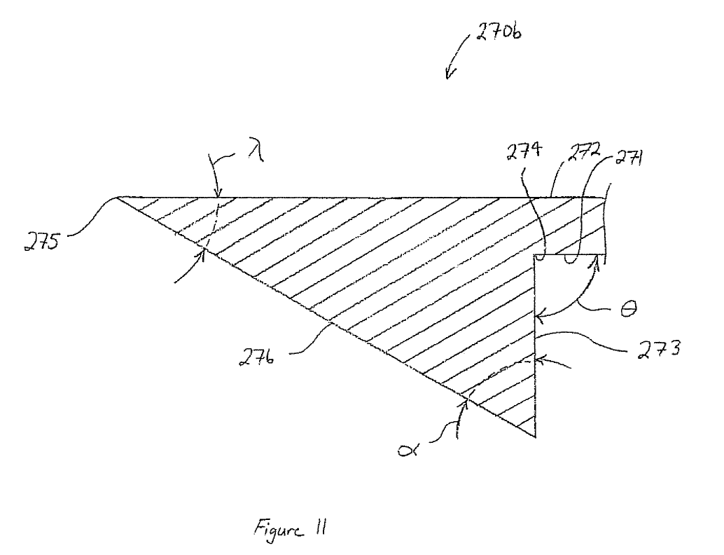

As previously described, in some embodiments, it may be desirable to retrieve and remove excised portions of the ligamentum flavum 26 from the distal cutting end 101 of tissue excision device 100. Referring now to FIGS. 9 and 10 an embodiment of a tissue retrieval device 200 is illustrated. Tissue retrieval device 200 comprises a cylindrical or tubular body 210 and a distal end or portion 260 extending from body 210. Body 210 and distal portion 260 are coaxially aligned, sharing the same central axis 250. In this embodiment, a through bore 240 is provided in body 210. Thus, body 210 has an inner surface 211 at a radius R₁ and an outer surface 212 at a radius R₂ as best seen in FIG. 10.

In the embodiment shown in FIGS. 9 and 10, body 210 and distal portion 260 have a substantially circular cross-section. However, it should be appreciated that in different embodiments, body 210 and distal portion 260 may have any suitable shape and cross-section including, without limitation, circular, oval, or polygonal. The geometry and shape of tissue retrieval device 200 is preferably compatible with the tissue excision device that it is used in conjunction with. In other words, tissue retrieval device 200 is preferably shaped and configured to be slidingly disposed within, and advanced within, the tissue excision device towards the excised portion of the ligamentum flavum cut and retained within the distal portion of the tissue excision device 100.

Referring still to FIGS. 9 and 10, in this embodiment, distal portion 260 comprises two prongs 270 extending from body 210 in a direction substantially parallel to axis 250. As used herein to describe this arrangement, prongs 270 may be described as extending “axially” or in an “axial” direction from body 210. However, it should be appreciated that in other embodiments, prongs 270 may be curved relative to axis 250. For instance, prongs 270 may be curved radially inward (i.e., towards central axis 250) to form pincers. In this embodiment, prongs 270 are opposite one another and uniformly spaced about 180° apart.

Each prong 270 has a fixed-end 270a integral with body 210, a free-end 270bdistal fixed-end 270a, an inner surface 271, and an outer surface 272. Each fixed-end 270ais fixed to body 210 such that prongs 270 do not move rotationally or translationally relative to body 210.

Inner surface 271 of each prong 270 is positioned substantially at the same radius as the inner surface 211 of body 210 (i.e., radius R₁) and is contiguous with inner surface 211 of body 210. In addition, outer surface 272 of each prong is positioned substantially at the same radius as outer surface 212 of body 210 (i.e., radius R₂) and is contiguous with outer surface 212 of body 210. As used herein, the term “contiguous” may be used to describe a relatively smooth connection without substantial breaks. Thus, each prong 270 is positioned substantially equidistant from axis 250. Although prongs 270 shown in FIG. 9 are each substantially the same and positioned substantially the same distance from axis 250, it should be understood that in other embodiments, one or more prongs (e.g., prongs 270) may be different in shape, length, geometry, and/or spaced at different distances from axis 250. As will be described in more detail below, a void or space 280 extending radially between prongs 270 accommodates excised tissue grasped by distal portion 260.

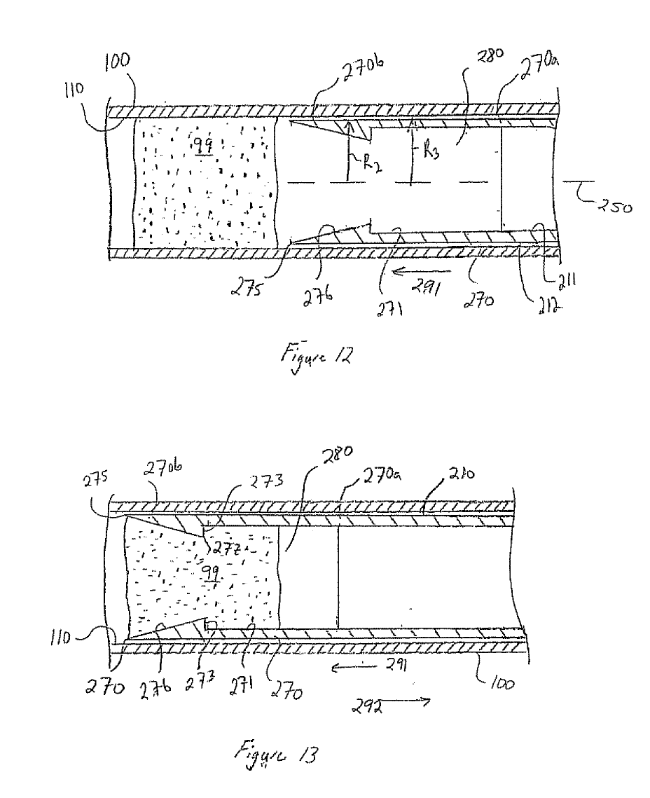

Referring now to FIGS. 10 and 11, a gripping surface 273 extends radially inward from inner surface 271 of each prong 270 proximal free-end 270b, thereby defining a shoulder 274. As best seen in FIG. 11, gripping surface 273 is oriented at an angle θ relative to inner surface 271 of each prong 270. In this embodiment, angle θ between inner surface 271 and gripping surface 273 is about 90°, however, in other embodiments, angle θ may be more or less than 90°. Further, although angle θ is substantially the same in each prong 270, it should be appreciated that angle θ may be the same or different for one or more prongs 270. As will be explained in more detail below, gripping surface 273 engages and grasps excised tissue segments that are retained within space 280 between prongs 270. To enhance the grasping of excised tissue segments within space 280, angle θ is preferably less than or equal to 90°.

Referring still to FIGS. 10 and 11, free-end 270b of each prong comprises a tip 275 that is sharpened as a result of a beveled surface 276 that extends radially inward between outer surface 272 and gripping surface 273. As best seen in FIG. 11, an angle λ between beveled surface 276 and outer surface 272 is preferably between 15° and 45°. In addition, beveled surface 276 and gripping surface 273 intersect at an angle α and form a corner or barb 277 that grasps tissue retained within space 280 between prongs 270. Angle α between beveled surface 276 and gripping surface 273 is preferably between 15° and 75°. It should be appreciated that the “sharpness” of barb 277 may be enhanced by decreasing angle α and decreasing angle θ. In some embodiments, one or more prongs 270 may include multiple barbs along its inner surface 211 between fixed-end 270a and free-end 270b that enhance the ability of the one or more prongs 270 to grasp tissue. Further, in some embodiments, a portion of inner surface 271 of one or more prongs 270 may be texturized or roughened to further improve the ability of prongs 270 to grasp excised tissue. Such texturing may include without limitation diamond knurling, sand blasting, bead blasting, plasma etching, or combinations thereof

The proximal end of tissue retrieval device 200 preferably includes a handle (not shown) to facilitate insertion and removal of tissue retrieval device 200 into and out of a tissue excision device. The handle may be constructed from any suitable material including machined metal or molded from plastic.

Referring now to FIG. 12, an excised tissue segment 99 is retained within a lumen or bore XX within tissue excision device 100. In particular, tissue excision device 100 is percutaneously inserted into the patient, with its distal cutting end (not shown) inserted first, and advanced toward the region of interest (e.g., enlarged ligamentum flavum). As previously described, tissue excision device 100 may be inserted and advanced through a cannula already positioned percutaneously through the patient. The distal cutting end of tissue excision device 100 is then positioned adjacent the region of interest. The distal cutting end is then employed to excise tissue segment 99 from the region of interest (e.g., excise a segment of enlarged ligamentum flavum). Excised tissue segment 99 is cut from the region of interest and disposed within a lumen or bore 110 of tissue excision device 100 adjacent the distal cutting end.

At this point, tissue segment 99 may be retrieved and removed from tissue excision device 100 by tissue retrieval device 200. Specifically, distal portion 260 of tissue retrieval device 200 is slidingly disposed coaxially within bore 110 and advanced through bore 110 in the direction of arrow 291 towards excised tissue segment 99. Tissue retrieval device 200 is preferably percutaneously inserted and advanced within tissue excision device 100. To ensure sliding engagement between tissue excision device 100 and tissue retrieval device 200, the inner radius R₃ of tissue excision device 100 defining bore 110 is preferably slightly greater than the outer radius R₂ of distal portion 260 and prongs 270. Thus, distal portion 260, including prongs 270, are advanced within bore 110 towards the segment of tissue segment 99.

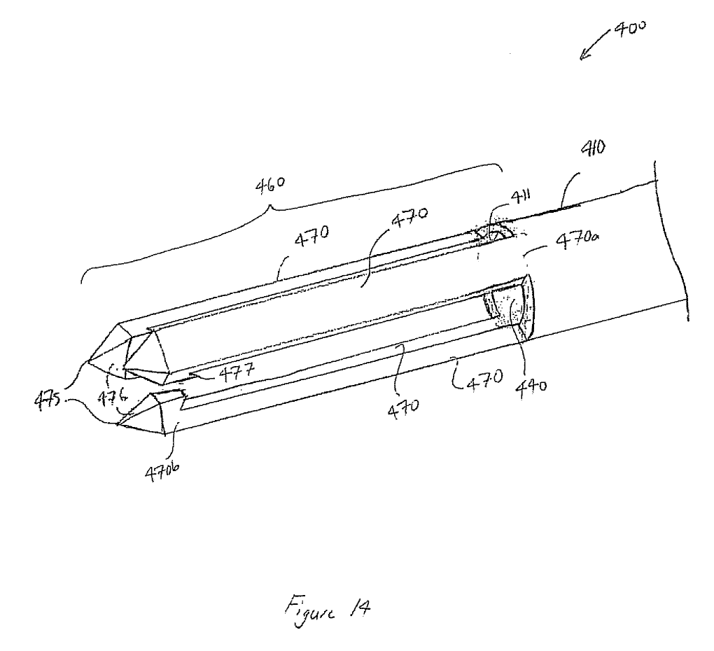

Referring now to FIGS. 12 and 13, sharpened tips 275 of prongs 270 first contact excised tissue segment 99 as distal portion 260 is advanced in the direction of arrow 291. Sharpened tips 275 and prongs 270 slide between tissue segment 99 and tissue excision device 100 as distal portion 260 is urged in the direction of arrow 291. In particular, beveled surfaces 276 engage and slide across excised tissue segment 99. As prongs 270 are advanced, excised tissue segment 99 moves towards and at least partially into space 280 between prongs 270.

Prongs 270 are preferably advanced until at least a portion of segment of excised tissue 99 contacts inner surface 271 and engages barbs 277 and gripping surface 273. Once barbs 277 and gripping surface 273 engage tissue segment 99, distal portion 260, including prongs 270, is retracted and removed from tissue excision device 100 in the direction of arrow 292. As distal portion 260 is withdrawn, barbs 277 and gripping surface 273 engage tissue segment 99, and pull excised tissue segment 99, thereby retrieving and removing excised tissue segment 99 from tissue excision device 100. Once tissue retrieval device 200 has been completely removed from tissue excision device 100, space 280 may be emptied by removing tissue segment 99 from between prongs 270. This process may be repeated to retrieve and remove additional tissue excised by tissue excision device 100.

In the manner described, excised tissue segment 99 may be retrieved and removed from a tissue excision device 100 by tissue retrieval device 200. The devices and methods described do not require the removal or repositioning of tissue excision device 100 within the region of interest. Rather, tissue excision device 100 is maintained in position while tissue retrieval device 200 is employed to grasp, retrieve, and remove excised tissue segment 99 from tissue excision device 100.

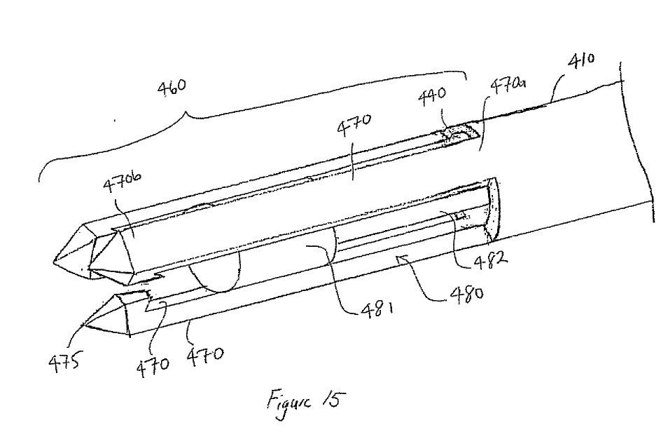

Referring now to FIGS. 14 and 15, an alternative embodiment of a tissue retrieval device 400 is illustrated. Tissue retrieval device 400 functions substantially the same as tissue retrieval device 200 previously described with the exception that tissue retrieval device 400 includes more than two prongs. Specifically, tissue retrieval device 400 comprises a body 410 and a distal portion 460 extending axially from body 410 that includes three prongs 470. Each prong 470 includes a fixed-end 470a integral with body 410 and a free-end 470b including a sharpened tip 475, beveled surface 476, and barbs 477 for grasping a segment of tissue.

In addition, in this embodiment, tissue retrieval device 400 also comprises a tissue ejector 480 slidingly disposed within a through bore 440 in body 410 (FIG. 15). Tissue ejector 480 includes a plunger 481 coupled to an ejection shaft 482. Plunger 481 and ejection shaft 482 slide axially within bore 440 of body 410. As depicted in FIG. 15, plunger 481 is shaped and configured to fit between prongs 470. Plunger 481 preferably slidingly contacts the inner surface 411 of body 410 and the inner surface 471 of prongs 470. Thus, plunger 481 preferably has an outer radius R₄ that is substantially the same or slightly less than the inner radius R₁ of body 410 and prongs 470. Once tissue retrieval device 400 has retrieved a tissue segment from a tissue excision device and has been completely removed from the tissue excision device, the tissue segment between prongs 470 may be removed from tissue retrieval device 400 by advancing ejector shaft 482 and plunger 481 toward the tissue segment. As plunger 481 engages the tissue segment, plunger 481 will force the tissue segment from between prongs 470, thereby ejecting the tissue segment from tissue retrieval device 400. In some embodiments, ejector 480 may be controlled by a multi-function tool. Embodiments of suitable multi-function tools for ejecting a tissue segment from a tissue retrieval device (e.g., tissue retrieval device 400) are disclosed in U.S. application Ser. No. 11/461,045, which is hereby incorporated herein by reference in its entirety.

Embodiments of the tissue retrieval device described herein (e.g., tissue retrieval device 200, tissue retrieval device 4300, etc.) may comprise any suitable material(s) for use in surgical instruments. Such materials include, without limitation, 400 series stainless steel, 17 series stainless steel, or 300 series stainless steel. The invention may be fabricated by any suitable means including, without limitation, machining, laser-cutting, electromechanical deposition, and electro-polishing. As previously described, in some embodiments, the inner surface of one or more of the prongs (e.g., prongs 270) may be textured to enhance engagement with the tissue. The textured surface may be diamond knurled, sand blasted, bead blasted, plasma etched, or media blasted.

Although the embodiments described herein disclose tissue retrieval devices (e.g., tissue retrieval device 200) having prongs (e.g., prongs 270) that are uniformly spaced apart, it should be understood that the prongs of a tissue retrieval device may likewise be spaced non-uniformly spaced.

In addition, the body and/or distal portion of the tissue retrieval devices described herein may be formed from any suitable hollow body including, without limitation, a hypotube, cannula, or catheter. Alternatively, the body, the distal portion, and/or any bores of the tissue retrieval devices described herein may be machined. As previously mentioned, the prongs (e.g., prongs 270) of the tissue retrieval devices described herein are preferably integral with the body (e.g., body 210) of the tissue retrieval device (e.g., tissue retrieval device 200). However, it should be understood that the prongs of a tissue retrieval device may alternatively be distinct components that are mechanically coupled to the body. In such alternative embodiments, the prongs may be coupled to the body by any suitable means including, without limitation, welding, pins, or combinations thereof.

Embodiments of devices (e.g., tissue excision devices, tissue retrieval devices, etc.), methods, and systems disclosed herein may take several forms and may be used according to the ILAMP method described above, or used according to alternative MILD procedures (e.g., MILD procedure schematically illustrated in FIG. 5). One such alternative MILD procedure is disclosed in U.S. application Ser. No. 11/193,581, which is hereby incorporated herein by reference in its entirety.

In addition, the methods and procedures disclosed herein may be facilitated by a kit for performing a spinal procedure (e.g., percutaneous decompression of enlarged ligamentum flavum). Such a kit preferably includes the basic components employed in one or more of the methods disclosed herein. For instance, in an embodiment, the kit preferably includes an insertion member (e.g., cannula) for accessing the epidural space, a contrast medium to create a safety zone, a tissue excision device to cut tissue segments, and an embodiment of the tissue retrieval device (e.g., tissue retrieval device 200, 400) to retrieve and remove the excised tissue segment from the tissue excision device. Depending on the application fewer or more components may be included in the kit.

While preferred embodiments have been shown and described, modifications thereof can be made by one skilled in the art without departing from the scope or teachings herein. The embodiments described herein are exemplary only and are not limiting. Many variations and modifications of the system and apparatus are possible and are within the scope of the invention. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims.

What is claimed is:

1. A device for retrieving an excised tissue segment comprising:
an elongate body having a central axis and an outer surface;
at least two prongs extending from the body in a direction substantially parallel to the central axis, wherein each prong includes a fixed-end coupled to the body, a free-end distal the body, an inner surface, and an outer surface substantially contiguous with the outer surface of the body; and
a space extending between the inner surfaces of the prongs that accommodates excised tissue.

2. The tissue retrieval device of claim 1 wherein the free-end of each prong includes a sharpened tip and at least one barb positioned along the inner surface of each prong.

3. The tissue retrieval device of claim 1 comprising two prongs generally opposite each other and spaced apart about 180°.

4. The tissue retrieval device of claim 1 wherein the prongs are positioned substantially equidistant from the central axis.

5. The tissue retrieval device of claim 1 wherein the outer surface of the body and the outer surface of each prong are each substantially at a radius R₁.

6. The tissue retrieval device of claim 5 wherein the body further comprises a central bore defining an inner surface of the body.

7. The tissue retrieval device of claim 6 wherein the inner surface of the body and the inner surface of each prong are substantially at a radius R₂.

8. The tissue retrieval device of claim 6 further comprising:
a tissue ejector including a plunger coupled to an ejector shaft, wherein the tissue ejector is slidingly received within the bore of the body; and
wherein the plunger slidingly engages the inner surface of the body and the inner surface of the prongs.

9. The tissue retrieval device of claim 1 wherein at least a portion of the inner surface of at least one prong is textured.

10. The tissue retrieval device of claim 9 wherein the textured portion of the inner surface of the at least one prong comprises diamond knurling, sand blasting, bead blasting, or plasma etching.

11. The tissue retrieval device of claim 1 wherein each prong is integral with the body.

12. The tissue retrieval device of claim 2 wherein the sharpened tip of each prong comprises a beveled surface extending radially inward from the outer surface of each prong, wherein an angle λ between the outer surface of each prong and the beveled surface of each prong is between 15° and 45°.

13. The tissue retrieval device of claim 12 wherein the free-end of each prong further comprises a gripping surface that extends radially inward from the inner surface of each prong, and wherein the gripping surface of each prong intersects the beveled surface of each prong to form the barb.

14. The tissue retrieval deice of claim 13 wherein an angle θ between the inner surface of each prong and the gripping surface of each prong is less than or equal to 90°.

15. The tissue retrieval device of claim 1 wherein at least one prong includes a second barb positioned along its inner surface between the free-end and the fixed-end.

16. The tissue retrieval device of claim 1 comprising three prongs extending from the body in a direction substantially parallel to the central axis, wherein each prong includes a fixed-end coupled to the body, a free-end distal the body, an inner surface, and an outer surface contiguous with the outer surface of the body.

17. The tissue retrieval device of claim 1 further comprising a handle coupled to the body opposite the at least two prongs.

18. A method for treating stenosis in a spine of a patient having a median plane, the spine including a spinal canal having a posterior surface, a dural sac and an epidural space between the posterior surface and dural sac, the location of the stenosis determining a region of interest in the spine, comprising:
a) inserting a tissue excision device percutaneously into the patient, wherein the tissue excision device comprises:
a distal cutting end; and
a through bore;
b) positioning the distal cutting end of the tissue excision device adjacent the region of interest;
c) excising a tissue segment from the region of interest with the tissue excision device, wherein the excised tissue segment is disposed within the bore of the tissue excision device;
d) inserting a tissue retrieval device percutaneously into the bore of the tissue excision device, the tissue retrieval device comprises:
an elongate body having a central axis and an outer surface; and
at least two prongs extending from the body in a direction substantially parallel to the central axis, wherein each prong includes a fixed-end coupled to the body, a free-end distal the body;
e) advancing the prongs towards the excised tissue segment within the bore of the tissue excision device;
f) grasping the excised tissue segment between the prongs of the tissue retrieval device; and
g) removing the excised tissue segment from the bore of the tissue excision device.

19. The method of claim 18 wherein a portion of the patient's ligamentum flavum occupies the region of interest, and wherein excising a tissue segment comprises inserting the cutting end of the tissue excision device into the ligamentum flavum in the region of interest and cutting at least a portion of the ligamentum flavum in the region of interest.

20. The method of claim 19 wherein removing the excised tissue segment comprises retracting the tissue retrieval device from the bore of the tissue excision device to remove at least a portion of the cut ligamentum flavum.

21. The method of claim 20 further comprising compressing the dural sac in the region of interest by injecting a fluid to form a safety zone and establish a working zone in the region of interest, the safety zone lying between the working zone and the dural sac.

22. The method of claim 18 further comprising emptying the excised tissue segment from the tissue retrieval device with a plunger coaxially disposed within a through bore in the body that slidingly engages the inner surface of the prongs.

23. A kit for performing a procedure on a spine, the spine including an epidural space containing a dural sac, the kit comprising:
a volume of a contrast medium adapted to be inserted into the epidural space by the insertion member and expanded so as to compress a portion of the thecal sac and provide a safety zone within the epidural space;
a tissue excision device; and
a tissue retrieval device.

24. The kit of claim 23 wherein the tissue retrieval device comprises
an elongate body having a central axis and an outer surface;
at least two prongs extending from the body in a direction substantially parallel to the central axis, wherein each prong includes a fixed-end coupled to the body, a free-end distal the body, an inner surface, and an outer surface substantially contiguous with the outer surface of the body; and
wherein the free-end of each prong includes a sharpened tip and at least one barb positioned along the inner surface of each prong.
